(19) United States
(12) Patent Application Publication
(10) Pub. No.: US 2010/0298242 A1
(43) Pub. Date: Nov. 25, 2010

(54) MASKING THE TASTE OF COMPOSITONS CONTAINING SALT

(76) Inventors: Reiner Postges, Frondenberg (DE); Richard Ammer, Iserlohn (DE); Joerg Breitkreutz, Haltern am See (DE); Dorothee Grueneberg Klinkowski, Dortmund (DE)

Correspondence Address: JENKINS, WILSON, TAYLOR & HUNT, P. A., 3100 Tower Blvd., Suite 1200, DURHAM, NC, 27707, US

(21) Appl. No.: 12/309,659

(22) PCT Filed: Jul. 26, 2006
(86) PCT No.: PCT/EP2006/064707

§ 371 Date: Mar. 16, 2010

(51) Int. Cl. A61K 38/05; A23L 2/60; A23L 2/52; A23L 1/236; C07C 313/22; A61K 31/54; C07C 229/34; C07D 291/06; A61P 43/00

(52) U.S. Cl. 514/21.91; 426/548; 426/74; 564/102; 514/222.5; 560/40; 544/2

(57) ABSTRACT

The invention relates to the use of sweeteners for masking the salty taste of compositions as well as compositions containing salt and defined sweeteners, the amount of sweeteners being suitable for masking the salty taste of the composition.

The present invention relates to the use of sweeteners for masking the salty taste of compositions and relates to compositions containing salt, which contain defined sweeteners, the quantity of sweetener being suitable to mask the salty taste of the composition.

### Sodium Chloride as an Active Ingredient in Therapy

Sodium chloride is used in diverse ways and used in various medicinal forms as an active ingredient for therapeutic purposes. In parenteral medicinal forms, such as, for example, infusion solutions, sodium chloride is used for electrolyte replacement. Sodium chloride is sprayed as an isotonic solution for inhalation in order to moisten the respiratory tracts.

Moreover, sodium chloride is an ingredient of glucose-electrolyte preparations, so-called oral rehydration salt (ORS) solutions. ORS solutions are applied orally for electrolyte and volume replacement. The preparations are packed portion-wise in sachets as a powder mixture. Before use, the powder is completely dissolved in a prescribed quantity of water. Finished preparations are, for example, Santalyt®, Elotrans®, Infectodiarrstop® or Oralpädon® 240. The preparations are frequently prescribed in paediatric therapy in order to ensure rehydration and an adequate electrolyte balance in diarrhoea disorders.

A drawback of these preparations is that, owing to the presence of sodium chloride, an aqueous ORS solution tastes salty. Further constituents are, inter alia, potassium chloride, which reinforces the salty taste of the solutions. The salty taste may lead to compliance problems. One aim in the development of pharmaceutical agents should be to make the taking of pharmaceutical agents as pleasant as possible. A masking of the salty taste should be aimed for pharmaceutical agents with a definite salty taste in order to avoid compliance problems.

### New WHO Guideline for ORS Solutions

The World Health Authority (WHO) issued a new guideline on ORS solutions in 2002. Because of new clinical study results, the concentrations of Na⁺, Cl⁻ and glucose have been reduced. As a consequence, the total osmolarity of the solutions has been reduced from 311 mosmol/l to 245 mosmol/l. Table 1 lists the old and new WHO recommendations from 1969 and 2002.

TABLE 1
Old and new WHO guidelines on the composition of ORS solutions

| Electrolyte/glucose | Old (1969) | | New (2002) | |
|---|---|---|---|---|
| Na⁺ | 90 | mmol/l | 75 | mmol/l |
| K⁺ | 20 | mmol/l | 20 | mmol/l |
| Cl⁻ | 80 | mmol/l | 65 | mmol/l |
| Glucose | 111 | mmol/l | 75 | mmol/l |
| Citrate³⁻ | 10 | mmol/l | 10 | mmol/l |
| Osmolarity | 311 | mosmol/l | 245 | mosmol/l |

Table 2 lists the molar fractions of exemplary preparations.

TABLE 2
Molar fractions of the components in finished pharmaceutical agents

| Constituent | Preparation A | | Preparation B | | Preparation C | |
|---|---|---|---|---|---|---|
| Na⁺ | 60 | mmol/l | 60 | mmol/l | 60 | mmol/l |
| K⁺ | 20 | mmol/l | 20 | mmol/l | 20 | mmol/l |
| Cl⁻ | 50 | mmol/l | 60 | mmol/l | 60 | mmol/l |
| Glucose | 111 | mmol/l | 90 | mmol/l | 90 | mmol/l |
| Citrate³⁻ | 10 | mmol/l | 10 | mmol/l | 10 | mmol/l |
| Total | 251 | mosmol/l | 240 | mosmol/l | 240 | mosmol/l |
| Osmolarity | | | | | | |

The European Society of Paediatric Gastroenterology and Nutrition (ESPGAN) also recommend a hypotonic composition for ORS solutions with 60 mmol/l sodium. The osmolarity should be in the region of 200 to 250 mosmol/l. The ESPGAN recommends a smaller maximum limit for the osmolarity than the WHO.

### Addition of Sweeteners to ORS Solutions

It is known to add sweetening agents to compositions containing salt, such as ORS solutions, for example. Sweetening agents include sugar, sugar alcohols and sweeteners. Sugars contribute to the energy balance of the body. The energy value of one gram of saccharose is 16.8 kJ. Sugars promote caries of the teeth. Insulin is required for the metabolisation of most sugars. They contribute to the total osmolarity of a solution. The sweetening power of sugar is small in comparison to sweeteners. Saccharose has a sweetening power of one. The sweetening power of each sweetening agent is related to the value one of saccharose. The sugar alcohols include sorbitol, maltitol, maltitol syrup, mannitol, isomalt, lactitol and xylitol. They are similar to sugars in taste. The sweetening power compared to saccharose is lower in all sugar alcohols. Sugar alcohols hardly contribute to the energy balance of the body. No insulin is consumed for metabolisation. The development of caries is not promoted. If sugar alcohols are taken in large quantities, diarrhoea and bloating may occur.

Sweeteners differ from sugars and sugar replacements with respect to many points. Regardless of the substantially higher sweetening power compared to sugars and sugar alcohols, sweeteners do not have an influence on the insulin level or on the digestion system or dental health. No insulin is consumed. No diarrhoea is produced. No caries occurs. Sweeteners have practically no calories influencing the energy balance. Currently, eight sweeteners are permitted in the European Union in food law. The sweeteners are listed in Table 3. An E number is provided for each sweetener and an ADI value is defined. The “acceptable daily intake” (ADI) value gives the quantity in milligrams per kilogram of body weight (KG) of the substance, which can be taken daily for life without incurring damage.

TABLE 3
Sweeteners permitted in the European Union

| | Sweetening | | |
|---|---|---|---|
| Sweetener | power | E number | ADI value/kg KG |
| Acesulfame potassium | 200 | E 950 | 15 mg |
| Aspartame | 200 | E 951 | 40 mg |
| Aspartame-acesulfame | 350 | E 962 | 40 mg/15 mg |
| salt | | | |
| Sodium cyclamate | 30 | E 952 | 11 mg |
| Neohesperidin | 1000 | E 959 | 5 mg |
| dihydrochalcone | | | |
| Saccharin sodium | 500 | E 954 | 5 mg |
| Sucralose | 600 | E 955 | 15 mg |
| Thaumatin | 3000 | E 957 | 5 mg |

The sweeteners are very heterogeneous with respect to their structure. They may be artificially produced compounds or compounds obtained from natural products. Artificial sweeteners are, for example, aspartame, acesulfame-K, Na-cyclamate or saccharin-Na. They are frequently present in a salt form in order to increase solubility in water. The aspartame-acesulfame salt consists of 64% aspartame and 36% acesulfame. A synergy in an increase of the sweetening power relative to the individual materials is achieved by the combination of the sweeteners. In the human organism, the aspartame-acesulfame salt is split into its original components aspartame and acesulfame. Acesulfame is eliminated unchanged via the kidneys. Aspartame is metabolised in the body. Aspartame is a phenylalanine source. If aspartame is contained in a product, the safety indication “This product contains a phenylalanine source” has to be printed on the packaging. The aspartame-acesulfame salt is marketed under the trade name Twinsweet®. Other sweeteners are of natural origin. Neohesperidin dihydrochalcone is a flavonoid derivative made of the peel of citrus fruits. Thaumatin is obtained from the West African katem fruit Thaumatococcus daniellii. Thaumatin is a natural protein. Two further sweeteners, steviosid and neotam are currently being checked for approval. Steviosid is obtained from the leaves of the plant Stevia Rebaudina Bertoni. The plant comes from South America. Steviosid has a sweetening power of 100 to 150. Neotam is an aspartame derivative. It has better hydrolytic stability. The sweetening power is about 10,000.

All previously known compositions containing salt (sometimes also containing sweetener), for example ORS, have the disadvantage as mentioned above, however, that they sometimes involve a very unpleasant salty taste, and this makes use significantly more difficult in paediatrics, in particular.

To this extent, the present invention is based on the object of providing a means for masking the salty taste of compositions containing salt. The present invention is based on the further object of providing a composition containing salt without an unpleasant salty taste, i.e. a composition in which the salty taste is masked as completely as possible. It is additionally an object of the invention to mask the salty taste of a composition which corresponds to the new WHO guidelines for ORS solutions.

These objects are achieved by the subject of the independent claims. Preferred embodiments are given in the sub-claims.

The present invention is based on the surprising recognition that the use of at least one sweetener from the group of sodium cyclamate, aspartame or acesulfame potassium is suitable for masking the salty taste of a composition. Although the individual sweeteners have already been used individually or in combination in preparations containing salt, this took place purely for the purpose of sweetening the preparation. Nothing was hitherto known of the suitability for masking the unpleasant salty taste and the quantities of sweetener hitherto used were also not adequate for this purpose.

Without wanting to be bound to a theory, the hitherto most plausible explanation for the surprising effect of sodium cyclamate, aspartame or acesulfame potassium on the taste perception of salty substances is possibly an influencing or competition of the signal transmission from taste receptors. Because the receptors for the sodium cation and the respective anions are structurally very different from those of the relevant sweetener, a direct inhibitory reciprocal effect at the receptors is improbable. However, it could be the case that the substances released after the receptor stimulation (“second messengers”) of sweet and salty taste perception influence one another or that the brain can no longer distinguish between the two taste impressions because of similar signal transmitter substances.

Possibly, the stimulation of taste cells by sodium ions ensures a depolarisation of the cells resulting in an intracellularly increasing calcium concentration. The three sweeteners according to the invention activate a receptor which empties calcium stores by means of the messenger inositol triphosphate, and this also leads to an increase in the intracellular calcium concentration in the same cells.

To this extent, the present invention relates, according to a first aspect, to the use of at least one sweetener from the group of sodium cyclamate, aspartame or acesulfame potassium, for masking the salty taste of a composition.

It has been shown according to the invention that in particular a combination of more than one of these sweeteners entails advantageous, synergistic effects. The quantity of the individual sweetener can be disproportionately reduced by the use of a combination of sweeteners and therefore the recommended highest daily doses for the individual sweeteners can be adhered to without problems (even in paediatrics). The required masking of taste is retained. In the case of the composition, according to the claims, of the ORS solutions, the total osmolarity recommended by the WHO is not exceeded despite the addition of sweetener.

To this extent, according to a preferred embodiment, a mixture of two or three of the above-mentioned sweeteners is used for masking taste.

As already mentioned, some salt-containing preparations, which also contain sweeteners as the sweetening agent, already exist. These were, however, added in doses, which are not suitable for masking the salty taste. The inventors have now found that the above-mentioned sweeteners in particular produce a masking of taste when they are used above a defined concentration threshold.

This concentration of the sweetener is calculated according to a preferred embodiment by the formula:

relative sweetening power of the sweetener×mass of the sweetener[in mg]>5,000

By way of example, in the case of Na-cyclamate as the only added sweetener, the following relationship would be produced (see also Table 3):

30×quantity Na-cyclamate[mg]>5,000

Thus the quantity of Na-cyclamate, which at least has to be used in order to (on its own) achieve a masking of taste, >166.66 mg. This quantity is generally based on a salt content of the composition of about 0.77 g, in other words about 0.7 to 0.8 g. With a larger quantity of salt, the sweetener quantity has to be corresponding increased.

These salts are primarily the salts NaCl and KCl used in, for example, ORS or other electrolyte compositions. Another salt, the taste of which can be masked is, for example, sodium-3-hydroxybutyrate. In principle, the taste of all salts can be masked, insomuch as it is perceived as salty. This is not the case, for example, for sodium citrate, which produces only a sour taste perception. Understandably, no masking of the salty taste can be achieved here either. In the case of sodium benzoate, which is at the same time perceived as bitter and salty, the salty taste component can be masked but the bitterness is then perceived just as much or even increasingly.

According to the invention, Na-cyclamate can preferably be used in a concentration of 22 to 28, preferably 26% by weight, based on the salt content of the composition. This concentration only applies to application on its own (without the addition of further sweeteners).

For aspartame and acesulfame potassium, in the case of use on their own, a concentration of 3.3 to 4.5, preferably 3.9% by weight based on the salt content of the composition is produced.

As already stated above, it is particularly advantageous to use a mixture of two or three of the above-mentioned sweeteners, the concentration of the sweetener mixture based on the salt content being calculated as follows:

rel. sweetening power Na-cyclamate×mass Na-cyclamate[in mg]+

rel. sweetening power aspartame×mass aspartame[in mg]+

rel. sweetening power acesulfame-K×mass acesulfame-K[in mg]>5,000

based on a salt content of the composition of 0.7 to 0.8, preferably 0.77 g.

Preferred combinations are, in particular:
a) Na-cyclamate/acesulfame-K
b) Na-cyclamate/aspartame, or
c) acesulfame-K/aspartame.

These are particularly preferably used in the following concentrations (in % by weight), based on the salt content:
a) 3.2/3.2
b) 3.9/3.2
c) 2.6/2.6

As already mentioned at an earlier point, in particular the taste of NaCl and/or KCl are to be masked.

The composition, in which the sweetener(s) is used, is preferably a pharmaceutical composition, a foodstuff or a food supplement. The pharmaceutical composition is, in particular, an electrolyte composition (ORS). The foodstuff or the food supplement is preferably an electrolyte drink.

According to a second aspect, the present invention comprises a composition containing salt, which contains at least one sweetener from the group of sodium cyclamate, aspartame or acesulfame potassium, the quantity of sweetener being suitable to mask the salty taste of the composition.

The composition preferably contains a mixture of two or three sweeteners for masking the taste. The concentration of the sweeteners based on the salt content is calculated as given above by the formula:

relative sweetening power of the sweetener×mass of the sweetener[in mg]>5,000

based on a salt content of the composition of 0.7 to 0.8, preferably 0.77 g.

Na-cyclamate, aspartame and acesulfame potassium are preferably used in the composition in the concentrations given above.

A mixture of two or three of the sweeteners is preferably used in the composition, the concentration of the sweetener mixture based on the salt content being calculated as follows:

rel. sweetening power Na-cyclamate×mass Na-cyclamate[in mg]+

rel. sweetening power aspartame×mass aspartame[in mg]+

rel. sweetening power acesulfame-K×mass acesulfame-K[in mg]>5,000

based on a salt content of the composition of 0.7 to 0.8, preferably 0.77 g.

The composition preferably contains the following sweetener combinations:
a) Na-cyclamate/acesulfame-K
b) Na-cyclamate/aspartame, or
c) acesulfame-K/aspartame,
   preferably in the concentrations given above.

With respect to the salts contained in the composition, see also the above statements.

The composition according to the invention is preferably a pharmaceutical composition, a foodstuff or a food supplement.

The term “pharmaceutical composition”, as used herein primarily means a pharmaceutical agent, in which electrolytes (together with the sweeteners according to the invention) are present in a quantity, which is suitable for the respective treatment purpose, for example for the supportive treatment in the case of diarrhoea disorders. The composition, in this case, also contains additional constituents such as glucose (see below), but also taste correctives, flavourings etc.

The pharmaceutical composition is preferably an electrolyte composition, preferably an ORS (oral rehydration salt) preparation.

The composition is alternatively a (dietary) foodstuff or a food supplement in the form of an electrolyte drink.

A pharmaceutical composition may, in addition to the one or more sweeteners, have the following ingredients:

| Constituent | Concentration [g]/Single dose |
|---|---|
| Glucose | 3.56 |
| Sodium chloride | 0.47 |
| Potassium chloride | 0.3 |
| Disodium hydrogen citrate-1,5 hydrate | 0.53 |

The present invention will now be illustrated by the following examples and figures. In the figures:

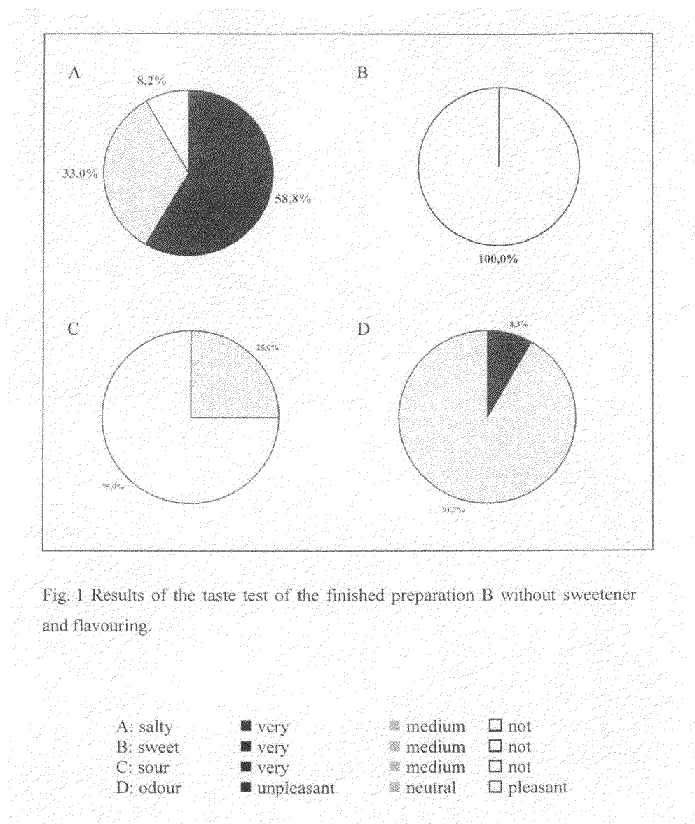

FIG. 1 shows results of the taste test of the finished preparation B without sweetener and flavouring.

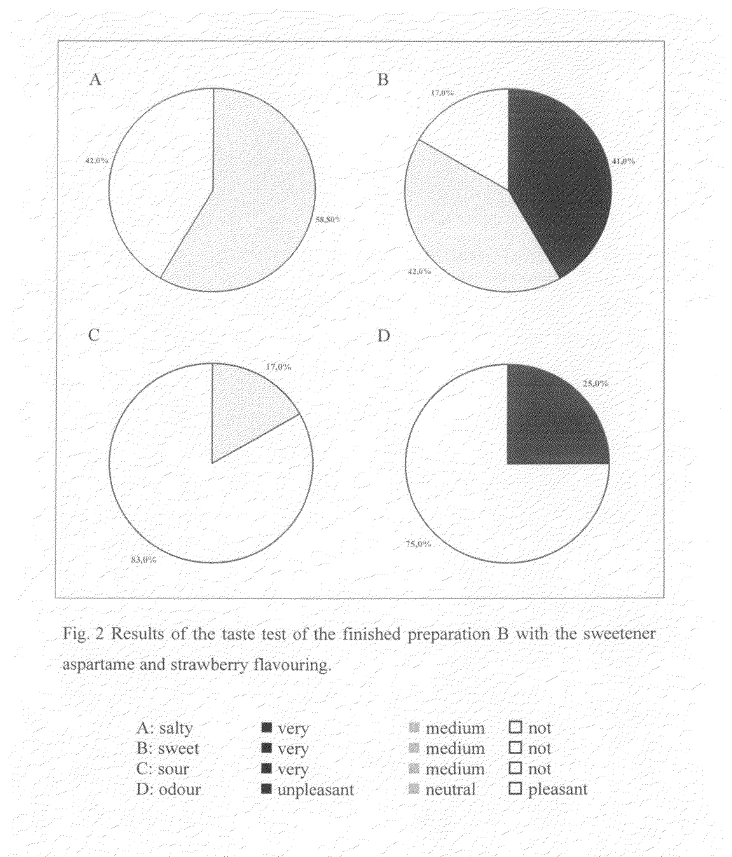

FIG. 2 shows results of the taste test of the finished preparation B with the sweetener aspartame and strawberry flavouring.

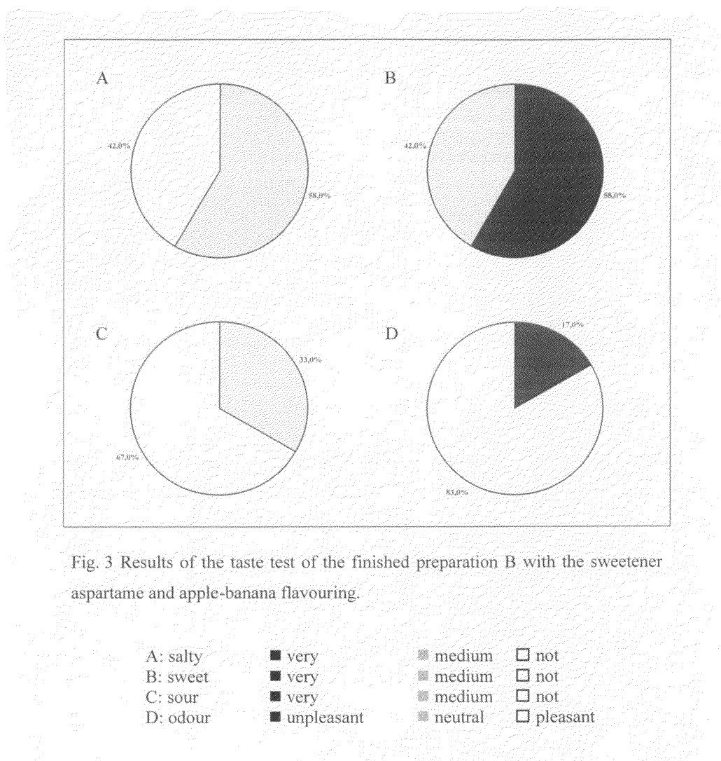

FIG. 3 shows results of the taste test of the finished preparation B with the sweetener aspartame and apple-banana flavouring.

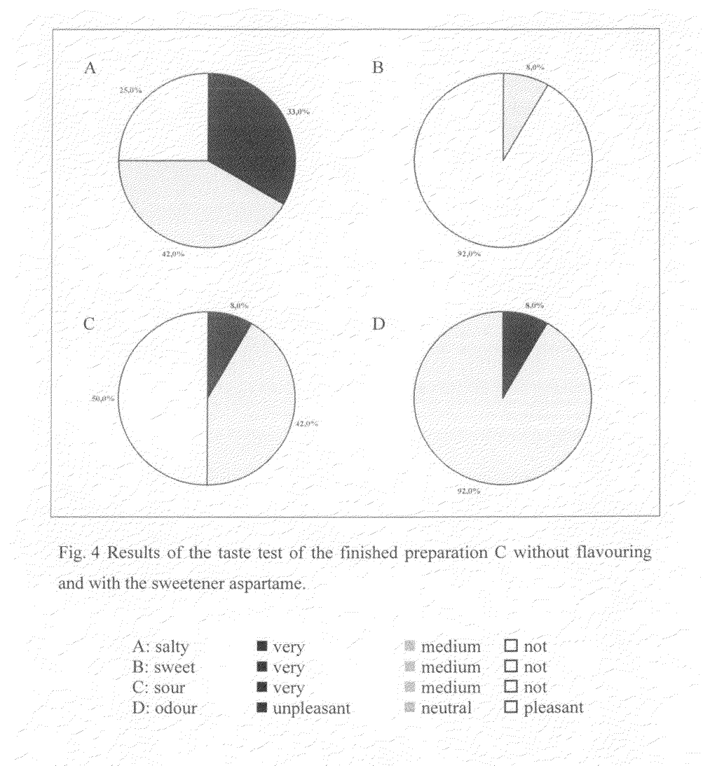

FIG. 4 shows results of the taste test of the finished preparation C without flavouring and with the sweetener aspartame.

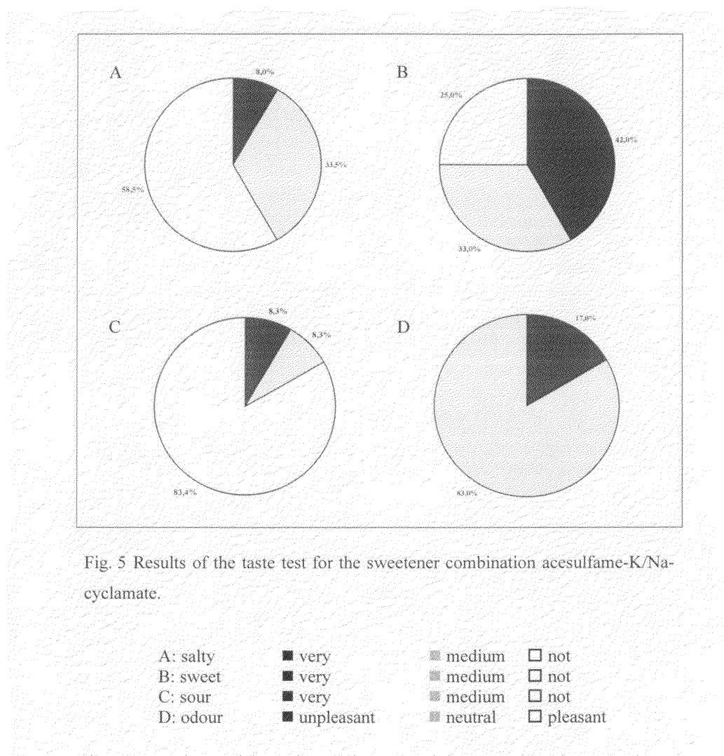

FIG. 5 shows results of the taste test for the sweetener combination acesulfame-K/Na-cyclamate.

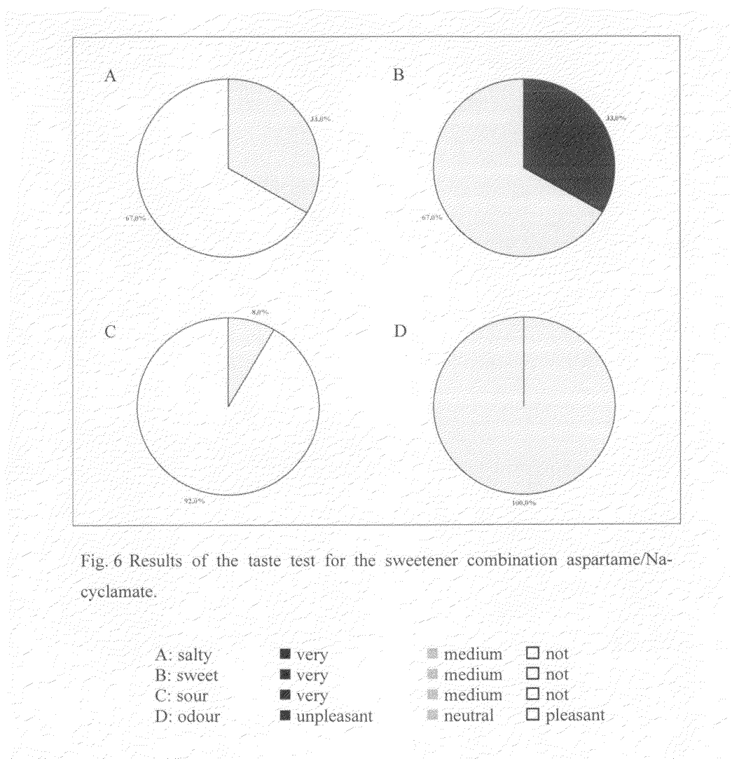

FIG. 6 shows results of the taste test for the sweetener combination aspartame/Na-cyclamate.

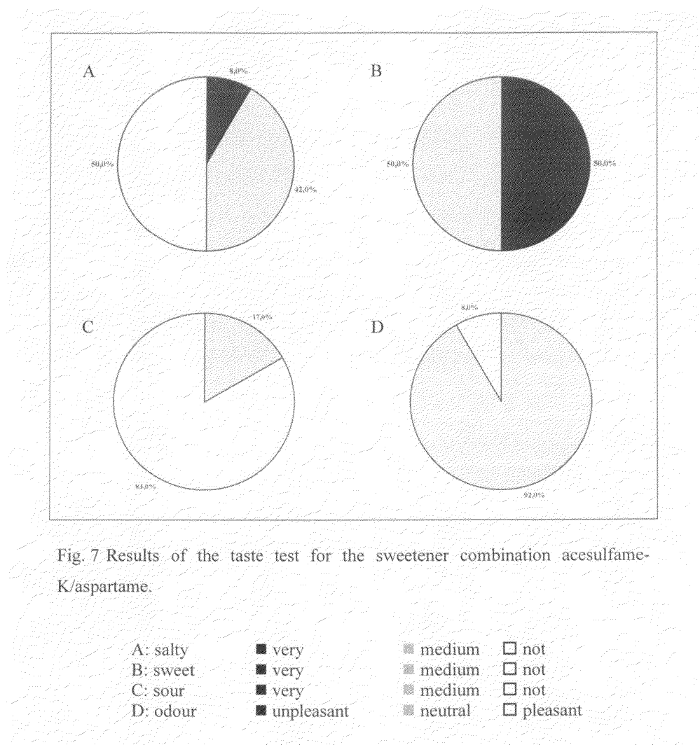

FIG. 7 shows results of the taste test for the sweetener combination acesulfame-K/aspartame.

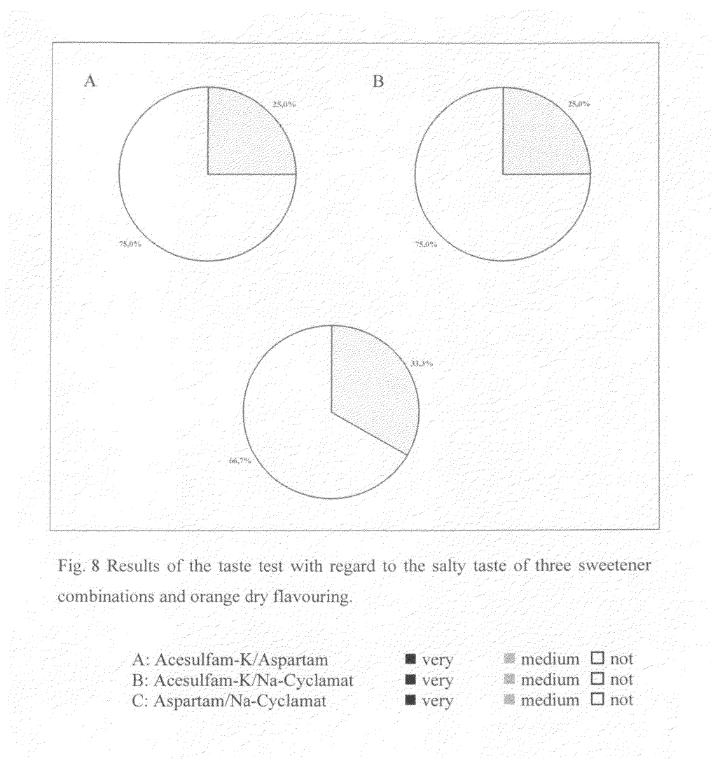

FIG. 8 shows results of the taste test with regard to the salty taste of three sweetener combinations and orange dry flavouring.

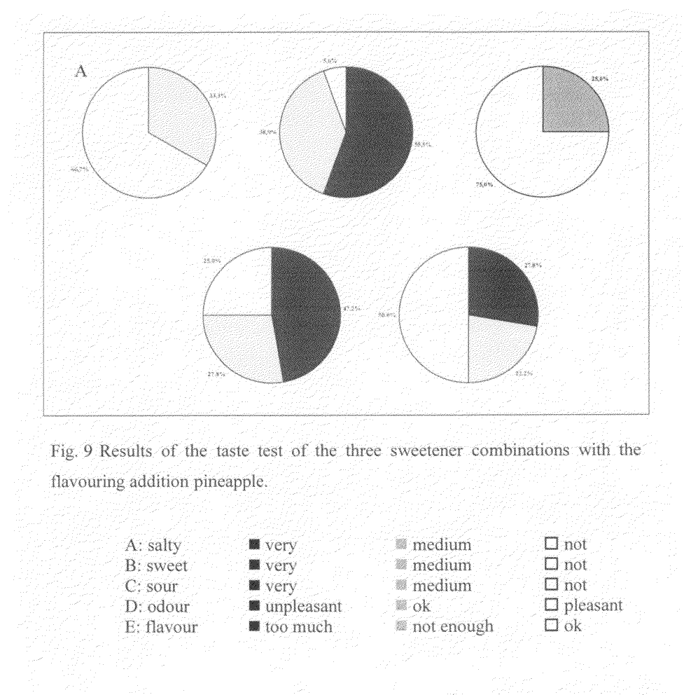

FIG. 9 shows results of the taste test of the three sweetener combinations with the flavouring addition pineapple.

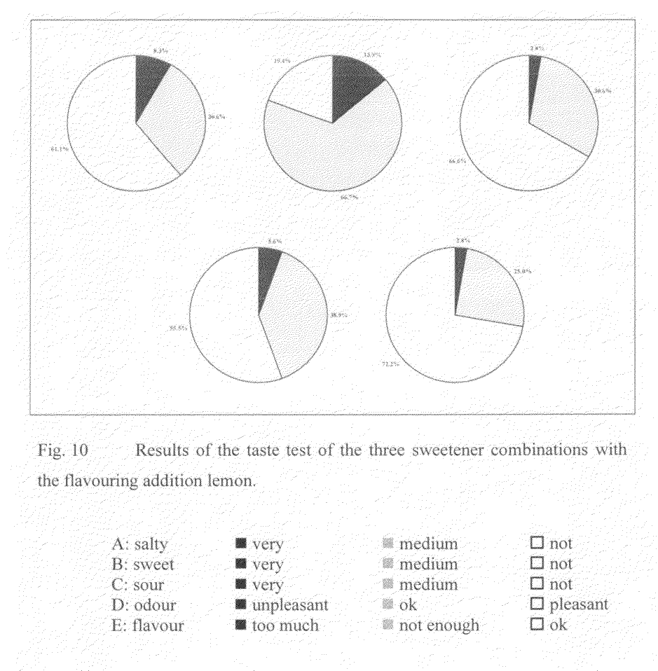

FIG. 10 shows results of the taste test of the three sweetener combinations with the flavouring addition lemon.

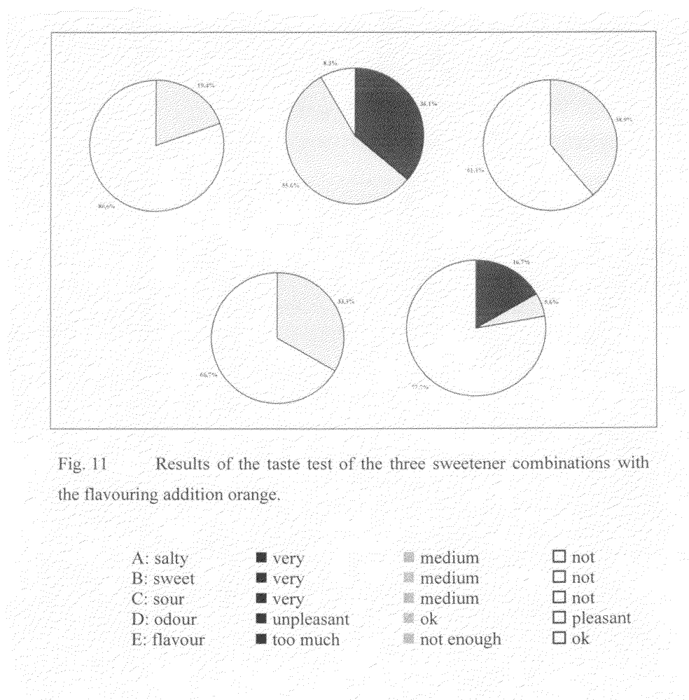

FIG. 11 shows results of the taste test of the three sweetener combinations with the flavouring addition orange.

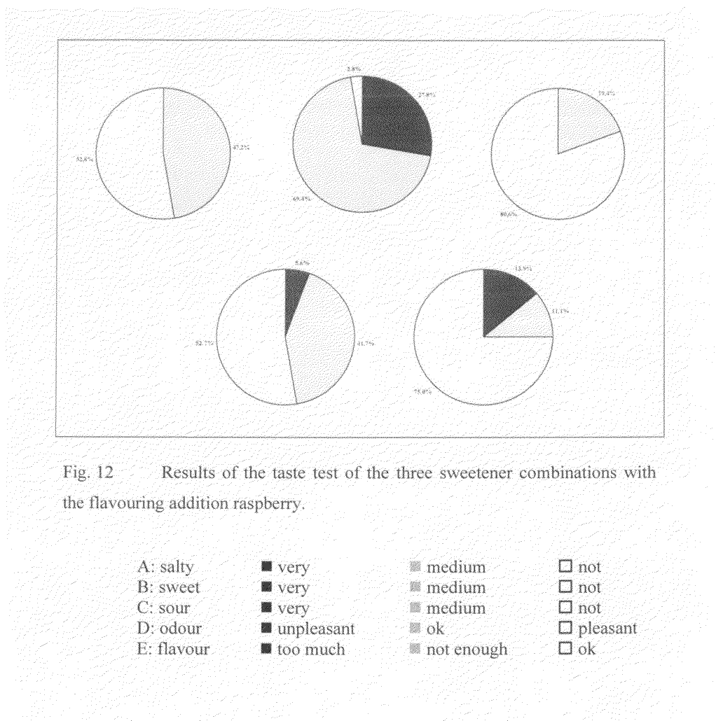

FIG. 12 shows results of the taste test of the three sweetener combinations with the flavouring addition raspberry.

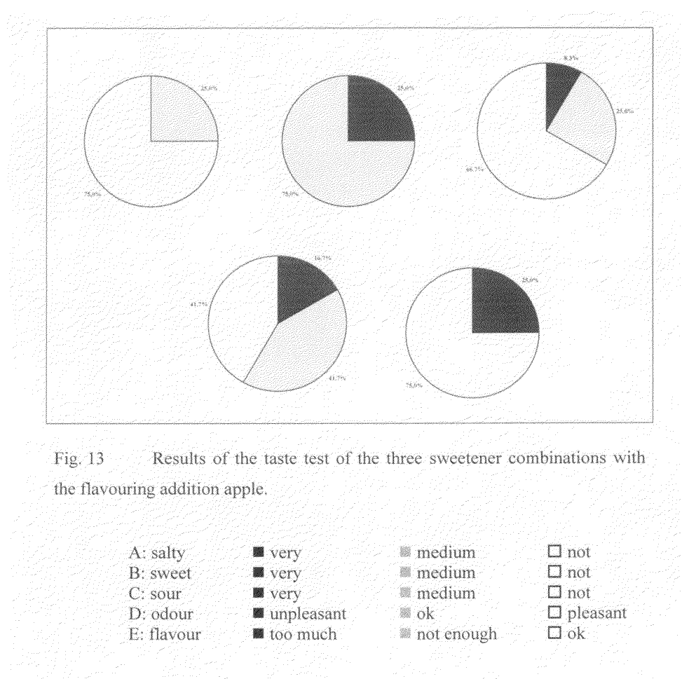

FIG. 13 shows results of the taste test of the three sweetener combinations with the flavouring addition apple.

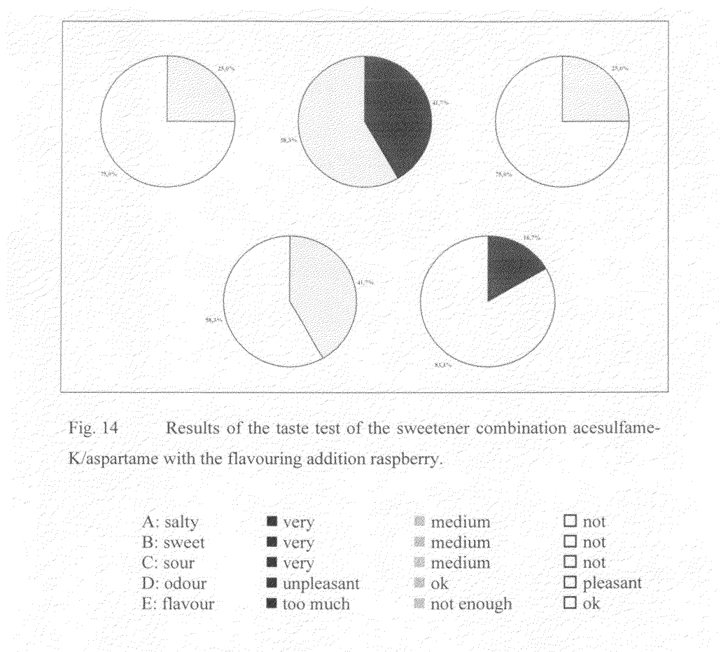

FIG. 14 shows results of the taste test of the sweetener combination acesulfame-K/aspartame with the flavouring addition raspberry.

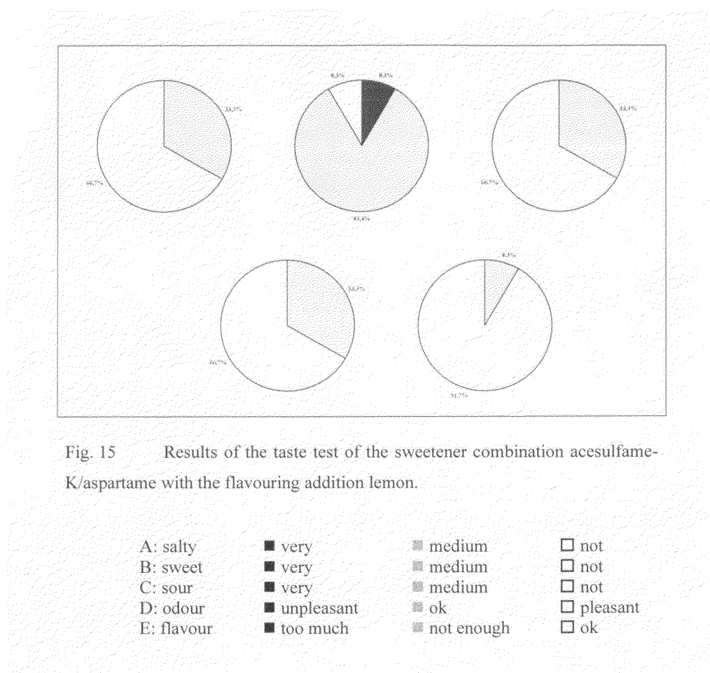

FIG. 15 shows results of the taste test of the sweetener combination acesulfame-K/aspartame with the flavouring addition lemon.

### EXAMPLES

Permitted sweeteners were used for a taste test on ORS solutions. Different concentrations of 10 to 200 mg of the sweetener Na-cyclamate were weighed into the solids mixture. Na-cyclamate is a sodium salt like NaCl. In comparison to other sweeteners, it has a low sweetening power. It was to be found out whether the salty taste of the ORS solution can be masked as a function of concentration by Na-cyclamate. The powder mixtures were weighed out according to the composition of the WHO and dissolved in water according to the instruction.

After organoleptic checking, no salty taste was perceived for the ORS solution with a concentration of 200 mg Na-cyclamate. A complete masking of taste had been achieved. The concentration of 200 mg Na-cyclamate was established as the standard concentration, with which a complete masking of taste can be achieved.

It was to be tested with the other sweeteners whether a comparable masking is possible. It was to be investigated whether by adding a sweetener with comparable sweetening, a masking of taste can also be achieved. The sweeteners were calculated to the standard concentration of the Na-cyclamate. The different sweetening power values of the sweeteners were used as the reference variable. Comparable concentrations could be calculated with a multiplication factor based on the sweetening power of the individual sweeteners. The concentrations are listed in Table 2.

TABLE 2
Concentrations of the sweeteners based on the
standard concentration of 200 mg Na-cyclamate

| | Sweetening | | Sweetener quantity |
|---|---|---|---|
| Sweetener | power | Factor | [mg] |
| Na-cyclamate | 30 | 1 | 200 |
| Acesulfame-K | 200 | 0.15 | 30 |
| Aspartame | 200 | 0.15 | 30 |
| Acesulfame-aspartame salt | 350 | 0.09 | 18 |
| Saccharin-Na | 500 | 0.06 | 12 |
| Sucralose | 600 | 0.05 | 10 |
| Neohesperidin | 1000 | 0.03 | 6 |
| dihydrochalcone | | | |
| Thaumatin | 3000 | 0.01 | 2 |

The calculated quantities of sweeteners were added to the ORS powder mixtures and dissolved in water. The following organoleptic test took place.

The salty taste of the ORS solutions could not be masked in a similar manner by any further sweetener concentration, as by the addition of 200 mg Na-cyclamate. The perception of the salty taste was different between the individual ORS solutions. The salty taste was perceived to a lesser extent by the addition of acesulfame-K, aspartame, the acesulfame-aspartame salt. Only a small to no improvement could be achieved by the remaining sweeteners. The sweet taste of thaumatin could only be perceived delayed by seconds. The salty taste of the ORS solution was immediately perceived and could also no longer be masked by the delayed sweet perception of the thaumatin.

After standardisation of the solutions to a comparable sweetness by the above-mentioned sweeteners, a comparable masking could be achieved.

### Sweetener Combinations

An aim of the present invention is to mask as completely as possible the salty taste of ORS solutions. The fact has been investigated that an addition of certain sweeteners leads to a masking or at least an improvement of the salty taste. A masking was achieved with the sweeteners Na-cyclamate, acesulfame-K, and aspartame. An attempt is to be made with the three sweeteners to improve the masking of the salty taste of ORS solutions.

An addition of sweeteners is not possible to unlimited extent (at least with regard to the current guidelines). ADI values (acceptable daily intake) were established for the sweeteners. Using the ADI values, maximum concentrations can be calculated, which may be applied daily by a substance without incurring damage for life. The sweetener addition to the ORS solutions is limited by the ADI values. According to the specialist information for preparation C, the therapeutic maximum dose is four sachets a day for babies and small children. As Na-cyclamate has an ADI value of 11 mg per kg of bodyweight, an addition of 200 mg Na-cyclamate per dose is too much. Table 3 lists the permissible highest daily doses for sweeteners, which may be applied daily. The calculation relates to the bodyweight of a child weighing 10 kg.

TABLE 3
Highest daily doses of sweeteners calculated to their ADI values

| | ADI value | | |
|---|---|---|---|
| | [mg/kg | Highest daily dose/10 kg | Highest |
| Sweetener | BW] | BW[mg] | dose/sachet [mg] |
| Na-cyclamate | 11 | 110 | 27.5 |
| Acesulfame-K | 15 | 150 | 37.5 |
| Aspartame | 40 | 400 | 100.0 |
| Saccharin-Na | 5 | 50 | 12.5 |

The sweetener addition is limited by the ADI values. An addition of 200 mg Na-cyclamate per dose of an ORS solution exceeds the permissible highest daily limit for a child weighing 10 kg by about twice the amount. The masking of the salty taste of ORS solutions is not possible by the addition of an individual sweetener. It is investigated whether a masking of the salty taste can be achieved by the addition of sweetener combinations to the constituents of an ORS solution.

When developing a pharmaceutical agent, as few auxiliary materials should be used as possible. Possible incompatibilities between the active ingredient and auxiliary material or intolerances in the patient to a constituent of the formulation are limited to a minimum.

In order to use as few sweeteners as possible, two sweeteners from Table 3 are combined with one another in each case. The result is six combinations. In order to mask the salty taste as successfully as possible, the concentrations of the sweeteners were selected to be close to the maximum daily highest limit, acesulfame-K 30 mg, Na-cyclamate 25 mg, saccharin-Na 10 mg. As aspartame has the comparatively highest ADI value, the maximum daily highest limit is the highest. With a acesulfame-K, aspartame in combination exhibits a synergistically increased sweetening power. As a result, the concentration with 40 mg for aspartame was only selected to be half as high as the maximally permissible daily highest limit. In order to ensure a comparability with the other sweeteners, the concentration of aspartame was also retained for the remaining combinations.

After organoleptic testing, the result of the investigation is that a complete masking of the salty taste of ORS solutions can be achieved by combinations of the sweeteners Na-cyclamate, acesulfame-K and aspartame. By means of the combinations with the sweetener saccharin-Na, an improvement of the taste could be achieved but the masking of the salty taste was not complete. In addition, saccharin-Na is suspected of promoting the growth of bladder carcinomas. In the further investigations work was only carried out with the sweeteners according to the invention, Na-cyclamate, acesulfame-K and aspartame.

Three sweetener combinations with the sweeteners Na-cyclamate, acesulfame-K and aspartame have been proven to be suitable for the masking of the salty taste of ORS solutions. Proceeding from the tested concentration ratios of the combinations it was investigated whether the same masking of taste can be achieved by a reduction in dose of the sweeteners. It was possible to reduce the concentration of the sweeteners. The final concentrations of the sweetener combinations for a masking of taste of the salty taste of ORS solutions are per dose:

| | | |
|---|---|---|
| Na-cyclamate/acesulfame-K | 25 mg/25 mg | sweetening power: 5750 |
| Na-cyclamate/aspartame | 25 mg/30 mg | sweetening power: 6750 |
| acesulfame-K/aspartame | 20 mg/20 mg | sweetening power: 8000 |

The sweetening power is different in all three combinations. The ability to mask a salty taste is highest for Na-cyclamate, followed by acesulfame-K and aspartame.

### Finished Preparations C and B

The finished preparations C and B with the flavours neutral, strawberry and apple-banana are combined according to the new WHO guideline (Table 4).

TABLE 4
Composition of preparation C and B

| Constituent | Concentration [g]/sachet |
|---|---|
| Glucose | 3.56 |
| Sodium chloride | 0.47 |
| Potassium chloride | 0.3 |
| Disodium hydrogen citrate-1,5 hydrate | 0.53 |

It emerges from the composition that a salty taste of an aqueous solution of the powder is to be expected by the addition of sodium chloride and potassium chloride in a total quantity of 0.77 g per dose. In the formulation of preparation C, the sweetener aspartame and highly disperse silicon dioxide are contained as further constituents. Only highly disperse silicon dioxide is additionally contained in preparation B neutral. In preparation B strawberry, the sweetener aspartame, highly disperse silicon dioxide, strawberry flavouring, malic acid and, as the colourant, beetroot dry extract (betanin, E 162) have been added. Preparation B apple-banana additionally contains the sweetener aspartame, highly disperse silicon dioxide, apple-banana flavouring, malic acid and the colourant carotin (E 160a).

With the exception of preparation B neutral, in the case of the finished preparations, sweeteners and flavourings are added to improve the taste. An aim of the present invention was to check whether the four finished preparations taste salty despite the additions for taste improvement.

It should be noted that both the preparation B and C contain sweetener concentrations which are far below the threshold concentrations according to the invention.

The taste perception was checked in a test attempt with 12 test subjects. The individual powder mixtures were prepared according to instructions for application directly before the tasting. The test took place in a randomised and double-blind manner. The evaluation criteria are the flavours salty, sweet and sour in the classifications of perception very-medium-not and the odour with the classifications pleasant-neutral-unpleasant.

FIG. 1: results of the taste test of the finished preparation B without sweetener and flavouring.

FIG. 2: results of the taste test of the finished preparation B with the sweetener aspartame and strawberry flavouring.

FIG. 3: results of the taste test of the finished preparation B with the sweetener aspartame and apple-banana.

FIG. 4: results of the taste test of the finished preparation C without flavouring and with the sweetener aspartame.

The colouring of the charts is based on a traffic light principle. The evaluation criterion which was classified as negative, for example a very salty taste of a solution, is shown in the colour red. The criterion which is considered satisfactory, for example a solution which tastes medium salty, is shown in the colour amber. The criterion which is considered positive, for example a solution which does not taste salty, is shown in the colour green. In the case of preparation B neutral, the salty taste of the solution was perceived most frequently and clearly of all the four preparations. 91.2% of the test subjects generally perceived a salty taste. 58.8% of the test subjects assessed the salty taste with “very”. Without the addition of taste correctives in preparation B neutral, a perception of the salty taste was expected in advance of the taste test. The result of the taste test confirms the expectation. Additions of sweeteners and flavourings are contained in the three remaining finished preparations. The salty taste was assessed less frequently and clearly in all three finished preparations than in preparation B neutral. A taste improvement can be achieved by flavourings and sweeteners. The extent varies.

Taste Testing of the ORS Solutions with the Addition of Sweetener Combinations in Comparison to Finished Pharmaceutical Agents

Sodium chloride and potassium chloride are contained inter alia as effective constituents in ORS solutions. An aqueous solution of the constituents is perceived as salty. For the finished pharmaceutical agents mentioned above, a perceivable salty taste has been shown in a test with test subjects. An aim of the present invention is to as completely as possible mask the salty taste of ORS solutions, as a salty taste is perceived as unpleasant and can lead to compliance problems in the therapy.

A complete masking could be achieved according to the invention with three sweetener combinations of the sweeteners Na-cyclamate, acesulfame-K and aspartame. The results were to be confirmed in a test with test subjects using 12 healthy test subjects. In order to be able to make a comparable statement, the above-mentioned finished preparations were also included in the test. The results of the ORS solutions with sweetener combinations are shown in FIGS. 5 to 11. Depending on the sweetener combination, the salty taste of the solutions was assessed as “not salty” by 50 to 67% of the test subjects. The finished preparations B neutral and C without an addition of flavouring were only described as “not salty” by 8% or 25%, respectively, of the test subjects. The addition of sweetener combinations increases the positive impression “not salty” by up to 59%.

FIG. 5: results of the taste test for the sweetener combination acesulfame-K/Na-cyclamate.

FIG. 6: results of the taste test for the sweetener combination aspartame/Na-cyclamate.

FIG. 7: results of the taste test of the sweetener combination acesulfame-K/aspartame.

### Osmolarity

The addition of sweeteners to ORS solutions changes the osmolarity of the solutions. The theoretical osmolarity is calculated at 240 mosmol/l owing to the effective constituents of the ORS solutions. The WHO recommends a total osmolarity of 245 mosmol/l. The sweeteners in the combinations and concentrations determined only increase the total osmolarity of the ORS solutions slightly.

TABLE 5
Osmolarity of the ORS solutions with sweeteners

| | | Theoretical | |
|---|---|---|---|
| | | osmolarity of | Measured |
| | Quantity | the total solution | osmolarity |
| Sweetener combination | [mg] | [mosmol/l] | [mosmol/l] |
| Acesulfame-K/aspartame | 20/20 | 241.3 | 234 |
| Acesulfame-K/Na- | 25/25 | 242 | 235 |
| cyclamate | | | |
| Aspartame/Na-cyclamate | 30/25 | 241.5 | 245 |

The addition of sweetening agents does not have a significant effect on the total osmolarity of the ORS solutions. Table 5 gives the measured osmolarities of the solutions. The recommendation of the WHO of optimally 245 mosmol/l is ensured.

Further sweetening agents were included in a taste testing. An addition of sugars and sugar alcohols has a substantially stronger effect on the total osmolarity of an ORS solution than the addition of sweeteners. Calculated to the standard concentration of 200 mg Na-cyclamate, the osmolarity values have different levels (Table 9). In addition to the total osmolarity of the effective constituents of the ORS solutions, the values are outside the recommended limits of the WHO recommendations. The quantities to be added are not to be recommended either in relation to the promotion of caries and a laxative effective. A combination of sugars or sugar alcohols does not lead to a significant improvement in the osmolarity.

TABLE 6
Osmolarity of sugars and sugar alcohols

| | | | | Theoretical |
|---|---|---|---|---|
| | Sweetening | | Calculated | osmolarity |
| Substance | power | Factor | quantity [mg] | [mosmol/l] |
| Lactose | 0.2 | 150 | 30000 | 440 |
| Mannitol | 0.4 | 75 | 15000 | 410 |
| Isomalt | 0.45 | 66.67 | 13334 | 195 |
| Glucose | 0.5 | 60 | 12000 | 333 |
| Sorbitol | 0.6 | 50 | 10000 | 274 |
| Maltitol | 0.9 | 33.33 | 6666 | 97 |
| Saccharose | 1 | 30 | 6000 | 88 |
| Xylitol | 1 | 30 | 6000 | 197 |
| Fructose | 1.2 | 25 | 5000 | 139 |
| Erythritol | approx. 2 | 15 | 3000 | 123 |
| Na-cyclamate | 30 | 1 | 200 | 10 |

A taste test was nevertheless carried out with the sugars and sugar alcohols. Complete dissolution took place in water and an organoleptic test took place with the calculated quantities. With maltitol, erithritol and saccharose a masking of the salty taste of the ORS solutions could substantially be achieved. The concentrations of substances were reduced to such an extent that the maximum limit of the osmolarity of 311 mosmol/l was adhered to. No masking of taste could be achieved by saccharose. For maltitol and erithritol, it was substantially not salty, but very sour and not sweet. The result is significantly different compared to the addition of sweetener combinations. The addition of sweeteners is to be preferred in each case with regard to side-effects, osmolarity and a complete masking of the salty taste.

### Addition of Flavouring

ORS solutions taste salty because of the ingredients NaCl and KCl. The European Medicines Agency (EMEA) recommend in their “Reflection paper: Formulations of choice for the paediatric population”, flavourings, which can be added to mask certain flavours. For a salty taste, these are the flavourings caramel, grapefruit, lemon, orange and vanilla. It should be checked whether a flavouring addition, in addition to the addition of a sweetener combination, more completely masks the salty taste of ORS solutions without the addition of a flavouring.

In a study, fruit juices were added for flavouring in order to make the taste of ORS solutions more pleasant. The ORS solutions were diluted in different ratios with apple or orange juice and orange lemonade. The taste was perceived as more pleasant owing to the dilution. The osmolarity of the solutions was increased by a multiple factor, however. The values neither corresponded to the requirements of the WHO with a maximum of 311 mosmol/l nor of the ESPGAN with a maximum of 250 mosmol/l. A dilution with juice or lemonade is not to be recommended. ORS solutions can only be flavoured by the direct addition of flavourings.

Dry flavourings may be exclusively added to a powder mixture for ORS solutions. The selection of a flavouring was oriented to the recommendations of the EMEA. Firstly, an orange dry flavouring was added to the powder mixture of an ORS solution. The concentration of the orange dry flavouring of 175 mg/dose was determined in advance. The sweetener combinations of acesulfame-K/aspartame, acesulfame-K/Na-cyclamate and aspartame/Na-cyclamate were added to the flavouring in the determined concentrations in each case. The flavoured ORS solutions were tested in the framework of the taste test at the sweetener concentrations. The results are shown in FIG. 8.

FIG. 8: results of the taste test of the salty taste of the three sweetener combinations and orange dry flavouring

The salty taste of the solutions with orange flavouring was assessed, depending on the sweetener combination, as “not salty” by 67 to 75% of the test subjects. The result is again better than the result of the test without a flavouring addition (50 to 67%). In a list of priority of the favoured solutions, of the ten test solutions, all three ORS solutions with a sweetener combination and orange flavouring in each case were evaluated at the first three places.

Taste testing of the ORS solutions with the addition of sweetener combinations and different flavourings in comparison to the preparation B apple-banana

Glucose-electrolyte solutions, so-called ORS solutions, contain NaCl and KCl inter alia as effective constituents. An active ORS solution tastes salty. After the addition of the sweetener combinations acesulfame-K/aspartame, acesulfame-K/Na-cyclamate and aspartame/Na-cyclamate, a masking of the salty taste could be substantially achieved. By adding an orange flavouring, the masking could be further improved. Further flavourings were to be tested in order to develop a mixture that is as optimal as possible for an ORS solution with a masked salty taste and pleasant taste. The flavourings lemon, orange, pineapple and raspberry were selected for a taste test. The concentrations of the flavourings for the test were determined in advance. The flavourings were tested with each sweetener combination in a taste test with 12 healthy test subjects. The finished preparation, preparation B apple-banana was included in the test as it was assessed as best in the previous tests of all finished preparations and is also flavoured. In total, 22 solutions were tasted. The results of the taste test are shown in FIGS. 9 to 13.

The salty taste of the ORS solutions is substantially masked by the addition of all the flavourings. The assessment “not salty” differs between 52.7 and 83.3% depending on the flavouring. The results relate to the assessment of the salty taste of all sweetener combinations of each individual flavouring to determine a preference for one flavouring.

FIG. 9: results of the taste test of all sweetener combinations with the flavouring addition pineapple.

FIG. 10: results of the taste test of all sweetener combinations with the flavouring addition lemon.

FIG. 11: results of the taste test of all sweetener combinations with the flavouring addition orange.

FIG. 12: results of the taste test of all sweetener combinations with the flavouring addition raspberry.

FIG. 13: results of the taste test of the finished preparation B apple-banana.

The test subjects were requested to select a favourite mixture per flavouring group from the three different sweetener combinations and to name their three overall favourites of all 22 solutions at the end. Despite a total assessment of only 52.8% for “not salty” for the flavouring raspberry, the ORS solutions were assessed as best. There is no complete masking of the salty taste. The overall taste impression of the ORS solutions is decisive for a positive evaluation in contrast to an evaluation of an individual criterion. The overall favourites were evaluated with the aid of an accumulative score. The flavourings raspberry and lemon were assessed as best.

TABLE 10
Results of the flavouring taste test of the ORS solutions

| | | | | Priority | |
|---|---|---|---|---|---|
| | Not | Osmolarity | pH | lists | Score |
| Flavouring | salty | (mosmol/l) | value | place | (accumulative) |
| Raspberry | 52.8% | 235 | 5.32 | 1 | 20 |
| Lemon | 61.1% | 237 | 5.39 | 2 | 20 |
| Orange | 80.6% | 228 | 5.40 | 3 | 13 |
| Preparation B | 75% | 244 | 4.95 | 6 | 3 |
| apple-banana | | | | | |
| Pineapple | 66.7% | 233 | 5.36 | 7 | 3 |

The addition of flavourings to the effective constituents of the ORS solutions and the sweetener combinations does not lead to a rise in the osmolarity. The measured osmolarities of the solutions are all in the range of the WHO recommendation of 245 mosmol/l. They are overall slightly lower (Table 10). The pH of the ORS solutions is in the slightly acid range at 5.40 in all our own formulations (Table 10). Of all the 22 solutions, the mixtures of the sweetener combinations acesulfame-K/aspartame with the flavourings raspberry or lemon were assessed as best. The individual results are shown in FIGS. 14 and 15. The ORS solution with raspberry flavouring and acesulfame-K/aspartame was assessed as being in the first place. The score is 14 points. The ORS solution with lemon flavouring and acesulfame-K/aspartame, with a score of 12 points, is in second place.

FIG. 14: results of the taste test of the sweetener combination acesulfame-K/aspartame with the flavouring addition raspberry

FIG. 15: results of the taste test of the sweetener combination acesulfame-K/aspartame with the flavouring addition lemon

The evaluations of the individual mixtures differ from the evaluation of the total flavouring groups. The evaluation of “not salty” at 75.0% compared to 52.8% of the total flavouring group is striking, in particular. In the mixture with lemon flavouring, the differences are even clearer: “not salty” 66.7% compared to 61.1%, “medium sweet” 83.4% compared to 66.7%, “not sour” 66.7% compared to 66.7%, “pleasant odour” 66.7% compared to 55.5% and “flavouring OK” 91.7% compared to 72.2%.

In total, the mixtures of sweeteners acesulfame-K and aspartame with the flavouring additions raspberry or lemon were assessed as best of all the formulations.

The optimal composition for ORS solutions is:

NaCl 0.47 g, KCl 0.3 g, glucose-monohydrate 3.56 g, Na-monohydrogen citrate-1,5-hydrate 0.53 g, acesulfame-K 20 mg, aspartame 20 mg, raspberry flavouring 150 mg or lemon flavouring 165 mg.

What is claimed is:

1. A method for masking the salty taste of a composition, the method comprising using at least one sweetener from the group of sodium cyclamate, aspartame or acesulfame potassium for masking the salty taste of a composition.

2. The method according to claim 1, wherein a mixture of two or three sweeteners is used for masking the taste.

3. The method according to claim 1, wherein the concentration of the sweeteners based on the salt content is calculated by the following formula:
relative sweetening power of the sweetener×mass of the sweetener[in mg]>5,000
based on a salt content of the composition of 0.7 to 0.8, preferably 0.77 g.

4. The method according to claim 3, wherein Na-cyclamate is used in a concentration of 22 to 28, preferably 26% by weight based on the salt content of the composition.

5. The method according to claim 3, wherein acesulfame potassium is used in a concentration of 3.3 to 4.5, preferably 3.9% by weight, based on the salt content of the composition.

6. The method according to claim 3, wherein aspartame is used in a concentration of 3.3 to 4.5, preferably 3.9% by weight, based on the salt content of the composition.

7. The method according to claim 1, wherein a mixture of two or three of the sweeteners according to claim 1 is used, wherein the concentration of the sweetener mixture based on the salt content is calculated as follows:
rel. sweetening power Na-cyclamate×mass Na-cyclamate[in mg]+
rel. sweetening power aspartame×mass aspartame[in mg]+
rel. sweetening power acesulfame-K×mass acesulfame-K[in mg]>5,000
based on a salt content of the composition of 0.7 to 0.8, preferably 0.77 g.

8. The method according to claim 1, wherein the following sweetener combinations are used:
Na-cyclamate/acesulfame-K
Na-cyclamate/aspartame, or
acesulfame-K/aspartame.

9. The method according to claim 8, wherein the following concentrations (in % by weight) based on the salt content of the composition are used in each case:
3.2/3.2
3.9/3.2
2.6/2.6

10. The method according to claim 1, wherein the taste of NaCl and/or KCl is to be masked.

11. The method according to claim 1, wherein the composition is a pharmaceutical composition, a foodstuff or a food supplement.

12. The method according to claim 11, wherein the pharmaceutical composition is an electrolyte composition.

13. The method according to claim 11, wherein the foodstuff or the food supplement is an electrolyte drink.

14. A salt-containing composition, which contains at least one sweetener from the group of sodium cyclamate, aspartame or acesulfame potassium, wherein the quantity of sweetener is suitable for masking the salty taste of the composition.

15. The composition according to claim 14, wherein a mixture of two or three sweeteners is used to mask the taste.

16. The composition according to claim 14 or 15, wherein the concentration of the sweeteners based on the salt content is calculated by the following formula:
relative sweetening power of the sweetener×mass of the sweetener[in mg]>5,000
based on a salt content of the composition of 0.7 to 0.8, preferably 0.77 g.

17. The composition according to claim 16, wherein Na-cyclamate is used in a concentration of 22 to 28, preferably 26% by weight, based on the salt content of the composition.

18. The composition according to claim 16, wherein acesulfame potassium is used in a concentration of 3.3 to 4.5, preferably 3.9% by weight, based on the salt content of the composition.

19. The composition according to claim 16, wherein aspartame is used in a concentration of 3.3 to 4.5, preferably 3.9% by weight, based on the salt content of the composition.

20. The composition according to claim 14, wherein a mixture of two or three of the sweeteners according to claim 14 is used, wherein the concentration of the sweetener mixture based on the salt content is calculated as follows:
rel. sweetening power Na-cyclamate×mass Na-cyclamate[in mg]+
rel. sweetening power aspartame×mass aspartame[in mg]+
rel. sweetening power acesulfame-K×mass acesulfame-K[in mg]>5,000
based on a salt content of the composition of 0.7 to 0.8, preferably 0.77 g.

21. The composition according to claim 14, wherein the following sweetener combinations are used:
Na-cyclamate/acesulfame-K
Na-cyclamate/aspartame, or
acesulfame-K/aspartame.

22. The composition according to claim 21, wherein the following concentrations (in % by weight) based on the salt content of the composition are used in each case:
3.2/3.2
3.9/3.2
2.6/2.6

23. The composition according to claim 14, wherein the salt comprises NaCl and/or KCl or consists thereof.

24. The composition according to claim 14, wherein the composition is a pharmaceutical composition, a foodstuff or a food supplement.

25. The composition according to claim 24, wherein the pharmaceutical composition is an electrolyte composition.

26. The composition according to claim 24, wherein the foodstuff or the food supplement is an electrolyte drink.

27. The composition according to claim 24, wherein the pharmaceutical composition is an ORS (oral rehydration salt) preparation.

28. The composition according to claim 14, which has the following ingredients:

| Constituent | Concentration [g]/Single dose |
|---|---|
| Glucose | 3.56 |
| Sodium chloride | 0.47 |
| Potassium chloride | 0.3 |
| Disodium hydrogen citrate-1,5 hydrate | 0.53 |
